(19) United States
(12) Patent Application Publication
(10) Pub. No.: US 2019/0069768 A1
(43) Pub. Date: Mar. 7, 2019

(54) CALCULATION SYSTEM

(71) Applicant: HOYA CORPORATION, Tokyo (JP)

(72) Inventor: Toru CHIBA, Tokyo (JP)

(73) Assignee: HOYA CORPORATION, Tokyo (JP)

(21) Appl. No.: 16/079,527

(22) PCT Filed: Jan. 10, 2017
(86) PCT No.: PCT/JP2017/000397

§ 371 Date: Aug. 23, 2018

(30) Foreign Application Priority Data
Feb. 26, 2016 (JP) 2016-035684

(51) Int. Cl. A61B 1/06; A61B 1/00; A61B 1/04; A61B 5/00; A61B 5/1455

(52) U.S. Cl. CPC A61B 1/0638; A61B 1/00186; A61B 5/14551; A61B 5/0084; A61B 1/042

(57) ABSTRACT

A calculation system is constituted by: one light source apparatus configured to irradiate illuminating light; a wavelength selection unit configured to select light of at least two specific wavelength regions included in the illuminating light; an image sensor configured to receive light from biological tissue, which is a subject, and output pixel signals corresponding to the received light; and a signal processing unit configured to perform predetermined signal processing on the pixel signals output from the image sensor. In this configuration, the signal processing unit calculates an index indicating a concentration of a predetermined biological substance included in the biological tissue, based on the pixel signals output from the image sensor according to the light of the at least two specific wavelength regions.

### TECHNICAL FIELD

The present invention relates to a calculation system.

### BACKGROUND ART

In recent years, an endoscope apparatus (spectral endoscope apparatus) including a spectral image capture function has been proposed. With this kind of spectral endoscope apparatus, information relating to a spectral characteristic of biological tissue such as a mucus membrane of a digestive organ (e.g., a reflection spectrum) can be obtained. It is known that the reflection spectrum of the biological tissue reflects information on the type and concentration of substances included near the surface layer of the biological tissue serving as the measurement target. Specifically, it is known that the absorption calculated using the reflection spectrum of the biological tissue is obtained by linearly superimposing the absorptions of multiple substances constituting the biological tissue.

It is known that the biological tissue of a lesioned part has a composition and component amount that are different from those of the biological tissue of a healthy part. In particular, many prior studies have reported that an abnormality at a lesioned part, which is represented by cancer or the like, is closely related to the state of the blood, and in particular, the states of the total blood amount and the oxygen saturation level. Here, a method of qualitatively and quantitatively analyzing two biological tissues of interest using spectroscopic characteristic amounts in the visible range of the biological tissues is often used in the field of spectral analysis chemistry. Accordingly, by comparing the spectral characteristic of blood in biological tissue including a lesioned part and that of biological tissue of only a healthy part, it is possible to determine whether or not some kind of lesioned part is included in the biological tissue.

A spectral image includes multiple pieces of image information captured with light of different wavelengths, and the larger the amount of wavelength information (number of wavelengths at which image information is acquired) included in the spectral image is, the more detailed the information of the biological tissue that can be acquired from the spectral image is. JP 2012-245223A (hereinafter written as “Patent Document 1”) discloses a configuration example of a spectral endoscope apparatus that acquires spectral images at a 5-nm wavelength interval in the wavelength region of 400 to 800 nm.

Also, JP 2013-099464A (hereinafter written as “Patent Document 2”) discloses an endoscope system including two light source apparatuses that emit illuminating light with mutually different wavelength bands. The two illuminating lights are emitted alternatingly to the subject each frame. In Patent Document 2, the oxygen saturation level is calculated using an image signal for when the subject is illuminated with the first illuminating light and an image signal for when the subject is illuminated with the second illuminating light.

### SUMMARY OF INVENTION

However, with the spectral endoscope apparatus of Patent Document 1, there is a problem in that it takes a long time to obtain information that is effective for diagnosis, since image analysis is performed after a large number of spectral images are obtained in 5-nm wavelength intervals. Also, in the endoscope system described in Patent Document 2, two light source apparatuses are needed, and image capture needs to be performed while switching between the two light source apparatuses each frame in order to calculate the oxygen saturation level, and therefore there is a problem in that the frame rate of the captured image decreases.

The present invention has been made in view of the foregoing circumstance, and aims to provide a calculation system according to which biological information such as an oxygen saturation level of hemoglobin included in biological tissue can be calculated without causing a decrease in the frame rate of a captured image.

According to an embodiment of the present invention, the calculation system includes: one light source apparatus configured to emit illuminating light; a wavelength selection unit configured to select light of at least two specific wavelength regions included in the illuminating light; an image sensor configured to receive light from biological tissue, which is a subject, and output pixel signals corresponding to the received light; and a signal processing unit configured to perform predetermined signal processing on the pixel signals output from the image sensor. In this configuration, the signal processing unit calculates an index indicating a concentration of a predetermined biological substance included in the biological tissue based on the pixel signals output from the image sensor according to the light of the at least two specific wavelength regions.

According to this kind of configuration, light of at least two wavelength regions is taken out by the wavelength selection means from the illuminating light emitted from the light source apparatus. An index indicating the concentration of the predetermined biological substance included in the biological tissue is calculated using the light of the at least two wavelength regions. Accordingly, there is no need to switch the illuminating light (light source) in order to calculate the index as in the prior technique, and when the subject is imaged, it is possible to prevent a decrease in the frame rate generated due to the switching of the illuminating light.

Also, according to an embodiment of the present invention, for example, the image sensor has three color filters for color image capture on light receiving surfaces of pixels, and wavelength regions of two of the three colors include the two specific wavelength regions respectively.

Also, according to an embodiment of the present invention, for example, the color filters include an R filter, a G filter, and a B filter, for which the wavelength regions of light that is transmitted therethrough are mutually different. In this case, one of the specific wavelength regions, which is transmitted by the G filter, includes a wavelength region defined by two predetermined isosbestic points of the hemoglobin, and the other of the specific wavelength regions, which is transmitted by the B filter, includes a wavelength region defined by two isosbestic points in a combination different from that of the two predetermined isosbestic points of the hemoglobin.

Also, according to an embodiment of the present invention, for example, the wavelength region of light that is transmitted by the R filter includes a first wavelength region of 600 nm or more, the wavelength region of light that is transmitted by the G filter includes a second wavelength region of 528 nm or more and 584 nm or less, and the wavelength region of light that is transmitted by the B filter includes a third wavelength region of 452 nm or more and 502 nm or less).

Also, according to an embodiment of the present invention, for example, the wavelength selection unit selects light of the first wavelength region, the second wavelength region, and the third wavelength region, which is included in the illuminating light.

Also, according to an embodiment of the present invention, for example, the wavelength selection unit is a single optical filter that selectively transmits or reflects light of the at least two specific wavelength regions.

Also, according to an embodiment of the present invention, for example, the wavelength selection unit includes at least two band-pass filters that respectively correspond to the at least two specific wavelength regions. In this case, the calculation system further includes a filter drive unit configured to selectively insert one of the at least two band-pass filters into an optical path of the illuminating light.

Also, according to an embodiment of the present invention, for example, the wavelength selection unit includes a first band-pass filter, a second band-pass filter, and a third band-pass filter. In this case, the first band-pass filter selectively transmits light of a wavelength region of 600 nm or more, the second band-pass filter selectively transmits light of a second wavelength region of 528 nm or more and 584 nm or less, and the third band-pass filter selectively transmits light of a third wavelength region of 452 nm or more and 502 nm or less.

Also, according to an embodiment of the present invention, for example, the wavelength selection unit is arranged between the light source apparatus and the biological tissue. In this case, the biological tissue is illuminated with the illuminating light selected by the wavelength selection unit as the light of the specific wavelength region.

Also, according to an embodiment of the present invention, for example, the wavelength selection unit is arranged between the biological tissue and the image sensor, and the wavelength selection unit selects light of the specific wavelength from reflected light reflected by the biological tissue. In this case, the image sensor receives the reflected light selected by the wavelength selection unit as the light of the specific wavelength region.

According to an embodiment of the present invention, a calculation system is provided, according to which biological information of biological tissue can be calculated without causing a decrease in the frame rate of a captured image.

### BRIEF DESCRIPTION OF DRAWINGS

FIG. 1 is a transmission spectrum of hemoglobin.

FIG. 2 is a graph obtained by plotting a relationship between a transmission light amount of blood in a wavelength region W2 and an oxygen saturation level.

FIG. 3 is a graph obtained by plotting a relationship between a transmission light amount of blood in a wavelength region W7 and an oxygen saturation level.

FIG. 4 is a block diagram showing a configuration of an electronic endoscope system of a first embodiment of the present invention.

FIG. 5 is a transmission spectrum of a color filter included in a solid-state image sensor.

FIG. 6 is a transmission spectrum of an optical filter.

FIG. 7 is a flowchart illustrating analysis processing according to the first embodiment of the present invention.

FIG. 8 shows examples of endoscope images generated by an electronic endoscope system according to the first embodiment of the present invention. FIG. 8(a) is an endoscope image and FIG. 8(b) is an oxygen saturation level distribution image.

FIG. 9 is a block diagram showing a configuration of an electronic endoscope system of a second embodiment of the present invention.

FIG. 10 is a front surface diagram of a rotary turret according to the second embodiment of the present invention.

FIG. 11 is a block diagram showing a configuration of an image capturing system of a third embodiment of the present invention.

### DESCRIPTION OF EMBODIMENTS

Hereinafter, embodiments of a calculation system of the present invention will be described with reference to the drawings. The calculation system of the present invention can be applied to an electronic endoscope system including an electronic endoscope or an image capture system including an image capture apparatus such as a digital video camera.

### First Embodiment

The first embodiment is an example in which the present invention is applied to an electronic endoscope system. The electronic endoscope system of the first embodiment is an apparatus that quantitatively analyzes the biological information of the subject (e.g., the oxygen saturation level or blood amount) based on an image (in the first embodiment, images of three wavelength regions, namely R, G, and B, which constitute one color image) captured with light of respective independent bands with different wavelengths, generates an image of the analysis results, and displays the image. In quantitative analysis of the oxygen saturation level or the like using the electronic endoscope system of the first embodiment described hereinafter, a property is used in which the spectral characteristic of blood in the visible range (i.e., the spectral characteristic of hemoglobin) changes continuously according to the oxygen saturation level.

### Principle of Calculating the Spectral Characteristic of Hemoglobin and the Oxygen Saturation Level

Before the specific configuration of the electronic endoscope system according to the embodiment of the present invention is described, the principle of calculating the spectral characteristic of hemoglobin in the visible range and the oxygen saturation level of the present embodiment will be described. Hemoglobin includes oxidized hemoglobin (HbO₂) and reduced hemoglobin (Hb), and the percentage made up by the oxidized hemoglobin is called the oxygen saturation level. The spectral characteristic of the hemoglobin changes according to the oxygen saturation level.

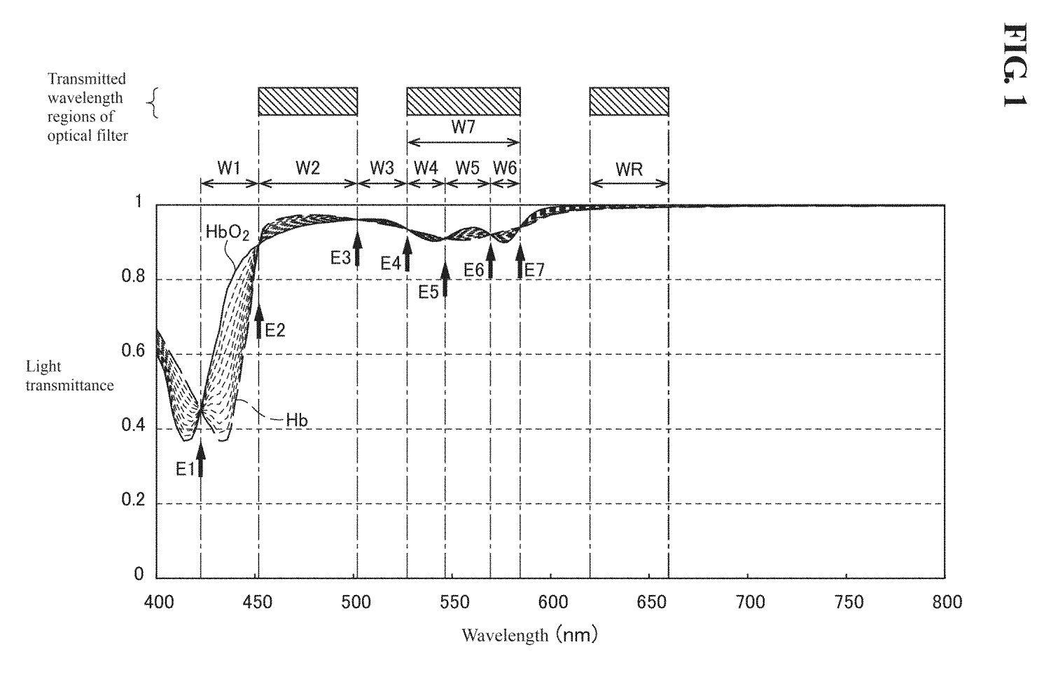

FIG. 1 shows a transmission spectrum of hemoglobin. The horizontal axis in FIG. 1 indicates the wavelength of light, and the vertical axis indicates light transmittance T. The waveform of the solid line in FIG. 1 is the transmission spectrum if the oxygen saturation level is 100% (i.e., oxidized hemoglobin) and the waveform of the long broken line is the transmission spectrum if the oxygen saturation level is 0% (i.e., reduced hemoglobin). Also, the short broken lines are transmission spectra of hemoglobin (a mixture of oxidized hemoglobin and reduced hemoglobin) at intermediate oxygen saturation levels (10, 20, 30, . . . , 90%).

Note that the absorption (absorbance) A of the hemoglobin is calculated based on the light transmittance T using the following Equation 1.

A=−log T Equation 1

As shown in FIG. 1, isosbestic points E1 (424 nm), E2 (452 nm), E3 (502 nm), E4 (528 nm), E5 (546 nm), E6 (570 nm), and E7 (584 nm) at which the light transmittance T (i.e., absorption A) is constant regardless of the oxygen saturation level appear in the transmission spectra of the hemoglobin. In the present specification, the wavelength region from the isosbestic point E1 to the isosbestic point E2 is defined as wavelength region W1, the wavelength region from the isosbestic point E2 to the isosbestic point E3 is defined as wavelength region W2, the wavelength region from the isosbestic point E3 to the isosbestic point E4 is defined as wavelength region W3, the wavelength region from the isosbestic point E4 to the isosbestic point E5 is defined as wavelength region W4, the wavelength region from the isosbestic point E5 to the isosbestic point E6 is defined as wavelength region W5, and the wavelength region from the isosbestic point E6 to the isosbestic point E7 is defined as wavelength region W6.

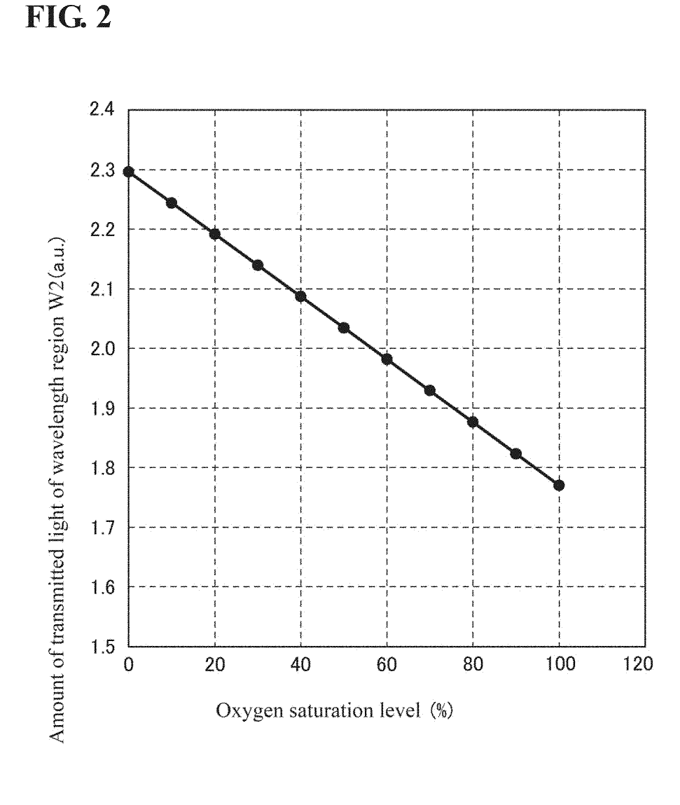

Between adjacent isosbestic points, the light transmittance T monotonically increases or decreases according to an increase in the oxygen saturation level. Also, between adjacent isosbestic points, the light transmittance T changes approximately linearly with respect to the oxygen saturation level. FIG. 2 is a graph obtained by plotting the relationship between the oxygen saturation level (horizontal axis) in the wavelength region W2 and the amount of light (vertical axis) that transmits through the hemoglobin. Note that the amount of transmitted light on the vertical axis is a value obtained by integrating the amount of transmitted light in the wavelength region W2. According to the graph of FIG. 2, it is understood that the amount of transmitted light decreases monotonically with respect to the oxygen saturation level in the wavelength region W2. Note that in the wavelength region W1 adjacent to the wavelength region W2, the amount of transmitted light increases monotonically with respect to the oxygen saturation level.

Giving attention to the wavelength region from the isosbestic point E4 to the isosbestic point E7 (i.e., the wavelength region that is continuous from wavelength region W4 to wavelength region 6; defined as “wavelength region W7” in the present specification), as shown in FIG. 1, the amount of transmitted light decreases monotonically according to an increase in the oxygen saturation level in the wavelength regions W4 and W6, but conversely, the amount of transmitted light increases monotonically according to an increase in the oxygen saturation level in the wavelength region W5. However, the inventor found that the amount by which the amount of transmitted light decreases accompanying an increase in the oxygen saturation level in the wavelength region W5 is approximately equal to the amount by which the sum of the amount of transmitted light increases accompanying an increase in the oxygen saturation amount in the wavelength regions W4 and W6, and in the wavelength region W7 as a whole, the amount of transmitted light is approximately constant, regardless of the oxygen saturation level. In other words, in the wavelength region W7 as a whole, the absorption A of the hemoglobin is approximately constant regardless of the oxygen saturation level.

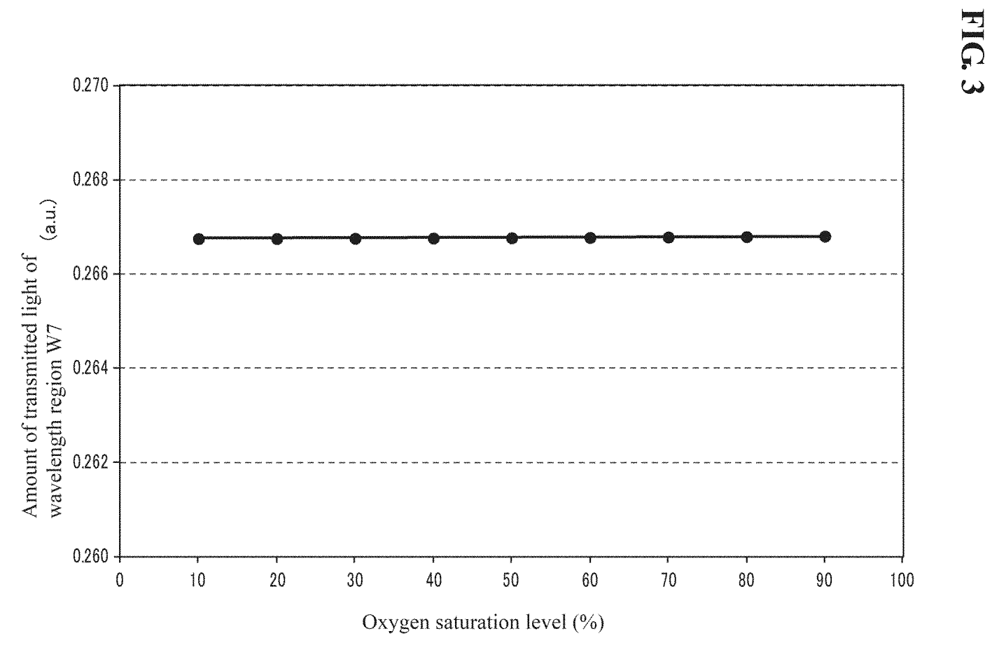

FIG. 3 is a graph obtained by plotting the relationship between the oxygen saturation level (horizontal axis) in the wavelength region W7 and the amount of light (vertical axis) that transmits through the hemoglobin. Note that the amount of transmitted light on the vertical axis is a value obtained by integrating the amount of transmitted light in the wavelength region W7. The average value of the amount of transmitted light was 0.267 (arbitrary unit), and its standard deviation was 1.86×10⁻⁵. According to the graph of FIG. 3, it is understood that the amount of transmitted light is approximately constant regardless of the oxygen saturation level in the wavelength region W7 as a whole.

Also, as shown in FIG. 1, in the wavelength region of approximately 600 nm and higher, the light transmittance T is high (the absorption A of light by the hemoglobin is small), and the light transmittance T hardly changes when the oxygen saturation level changes. For this reason, when a subject including hemoglobin (blood) is observed illuminated by white-colored light, the wavelength region of 600 nm and higher (e.g., the wavelength region of 600 to 660 nm, or 620 to 660 nm) can be used as a transparent region in which there is no absorption by the hemoglobin, and as a reference wavelength region for the transmitted light amount T (or absorption A). In the present embodiment, the wavelength region from the wavelength 620 nm to the wavelength 660 nm is defined as the wavelength region WR.

As described above, the amount of light that transmits through the hemoglobin in the wavelength region W2 decreases monotonically with respect to an increase in the oxygen saturation level, and the amount of light that transmits through the hemoglobin in the wavelength region W7 (wavelength regions W4 to W6) can be considered to be a constant value regardless of the oxygen saturation level. For this reason, based on the amount of light transmitted in the wavelength region W2 and the amount of light transmitted in the wavelength region W7, it is possible to obtain an index indicating the amount of hemoglobin (i.e., blood) in the subject (biological tissue) and an index indicating the oxygen saturation level of the blood. Accordingly, if the relationship between the blood amount and the index indicating the blood amount, and the relationship between the oxygen saturation level and the index indicating the oxygen saturation level are obtained in advance experimentally or through calculation, the blood amount and the oxygen saturation level can be estimated based on the values of the indices.

Note that if the illuminating light is emitted to the biological tissue and the biological tissue is observed based on the light reflected by the biological tissue, the larger the light absorption degree of the biological tissue resulting from the hemoglobin is (the smaller the light transmittance is), the smaller the reflection rate of the illuminating light by the biological tissue is. On the other hand, the smaller the absorbance of the hemoglobin is (the larger the light transmittance is), the larger the reflectance of the illuminating light by the biological tissue is. For this reason, by detecting the reflected light from the biological tissue, it is possible to calculate an index indicating the blood amount and an index indicating the oxygen saturation level.

### Overall Configuration of the Electronic Endoscope System 1

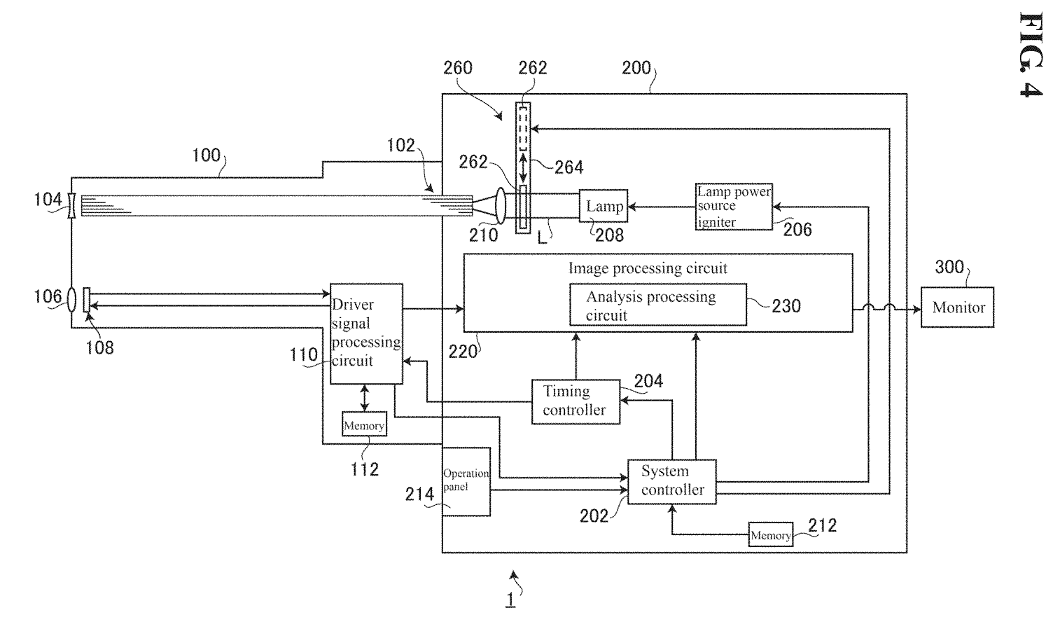

FIG. 4 is a block diagram showing a configuration of an electronic endoscope system 1 of a first embodiment of the present invention. As shown in FIG. 4, the electronic endoscope system 1 includes an electronic endoscope 100, a processor 200, and a monitor 300.

The processor 200 includes a system controller 202, a timing controller 204, an image processing circuit 220, a lamp 208, and an optical filter apparatus 260 that is an example of a wavelength selection unit. The system controller 202 executes various types of programs stored in the memory 212 and performs overall control of the electronic endoscope system 1. Also, the system controller 202 is connected to an operation panel 214. The system controller 202 changes the various operations of the electronic endoscope system 1 and parameters for the various operations according to an instruction from a user, which is input using the operation panel 214. The timing controller 204 outputs a clock pulse for adjusting the timing of operations of the units to circuits in the electronic endoscope system 1.

In the present embodiment, a lamp power source igniter 206 and the lamp 208 constitute an example of a light source apparatus. After being started up by the lamp power source igniter 206, the lamp 208 emits illuminating light L. The lamp 208 is a high-luminance lamp such as a xenon lamp, a halogen lamp, a mercury lamp, or a metal halide lamp, or is a white LED (Light Emitting Diode). The illuminating light L is mainly light (white light including at least the visible range) having a spectrum that extends from the visible range (or near-ultraviolet range) to the infrared range, which is invisible.

The optical filter apparatus 260 is arranged between the lamp 208 and a converging lens 210. The optical filter apparatus 260 includes a filter drive unit 264 and an optical filter 262 mounted on the filter drive unit 264. The filter drive unit 264 is constituted so as to be able to slide the optical filter 262 in a direction orthogonal to the optical path, between the position (solid line) on the optical path of the illuminating light L and a position (broken line) of being retracted from the optical path. Note that the configuration of the filter drive unit 264 is not limited to the description above, and for example, it is also possible to use a configuration in which the optical filter 262 is inserted into and removed from the optical path of the illuminating light L due to the optical filter 262 being rotated about a rotational axis located away from the center of gravity of the optical filter 262. The details of the optical filter 262 will be described later.

The electronic endoscope system 1 of the present embodiment is configured to be able to operate in three modes, namely a normal observation mode in which endoscopic observation is performed using the white light emitted from the lamp 208 as-is (or with the infrared component and/or ultraviolet component removed) as the illuminating light (normal light Ln), a special observation mode in which endoscopic observation is performed using filtered light Lf obtained by passing the white light through the optical filter 262 (or, with the infrared component and/or ultraviolet component further removed) as the illuminating light, and a baseline measurement mode for acquiring correction values to be used in the special observation mode. The optical filter 262 is arranged at a position retracted from the optical path in the normal observation mode, and is arranged on the optical path in the special observation mode.

The illuminating light L (filtered light Lf or normal light Ln) that has passed through the optical filter apparatus 260 is collected on the incident end surface of an LCB (Light Carrying Bundle) 102 by the converging lens 210, and is introduced into the LCB 102.

The illuminating light L introduced into the LCB 102 is transmitted by the TCB 102, emitted from the exit end surface of the LCB 102 arranged on the leading end of the electronic endoscope 100, and is irradiated to the subject via the light distribution lens 104. The returning light from the subject irradiated by the illuminating light L forms an optical image on the light receiving surface of the solid-state image sensor 108 via an object lens 106.

The solid-state image sensor 108 is a single-plate color CCD (Charge Coupled Device) image sensor having a Bayer pixel arrangement. The solid-state image sensor 108 generates and outputs an image signal by accumulating optical images formed by the pixels on the light receiving surface as charges corresponding to light amounts. The solid-state image sensor 108 includes on-chip color filters, namely an R filter that transmits red light, a G filter that transmits green light, and a B filter that transmits blue light, the color filters being formed directly on the pixels of the solid-state image sensor 108. The image signals generated by the solid-state image sensor 108 include an image signal R output from the pixels equipped with the R filter, an image signal G output from the pixels equipped with the G filter, and an image signal B output from the pixels equipped with the B filter.

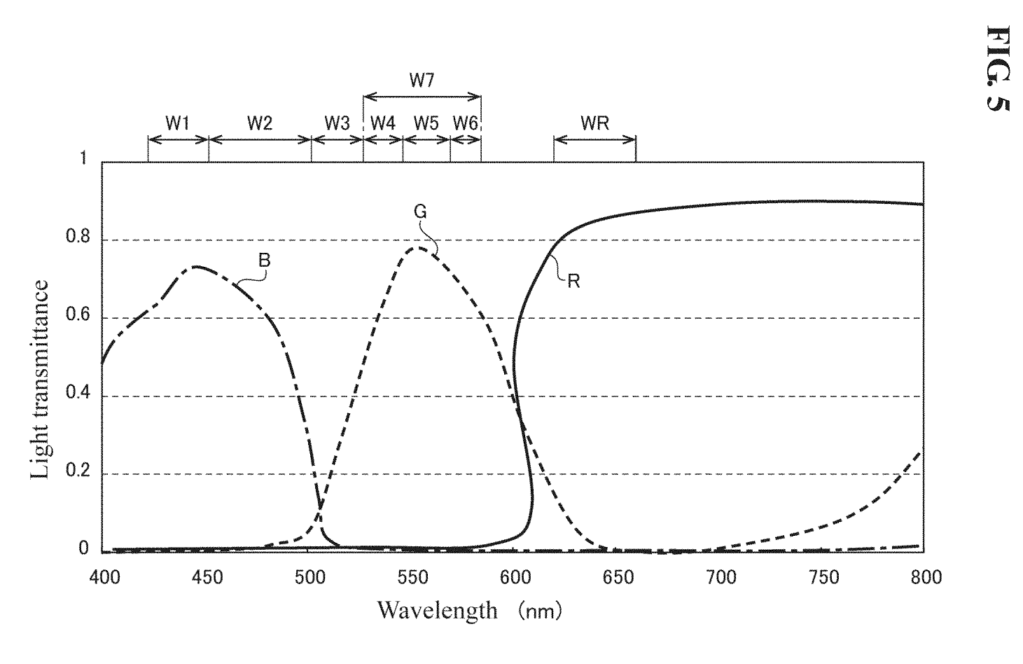

FIG. 5 shows transmission spectra of the R filter, G filter, and B filter of the solid-state image sensor 108. The horizontal axis in FIG. 5 indicates the wavelength, and the vertical axis indicates the light transmittance of each filter. The R filter is a filter that transmits light of a wavelength region of about 600 nm and more, including a wavelength region WR. The G filter is a filter that transmits light of the wavelength region of about 510 to 630 nm including a wavelength region W7. Also, the B filter is a filter that transmits light of the wavelength region of about 510 nm and less, including the wavelength regions W1 and W2. Also, as will be described later, the optical filter 262 has an optical characteristic of selectively transmitting only light of the wavelength regions WR, W7, and W2. Images of the light of the wavelength regions WR, W7, and W2 that has transmitted through the optical filter 262 are captured by the pixels equipped with the R filter, G filter, and B filter of the solid-state image sensor 108, and are output as the pixel signals R, G, and B.

Note that the solid-state image sensor 108 is not limited to a CCD sensor, and may be replaced with a CMOS (Complementary Metal Oxide Semiconductor) image sensor or another type of image capturing apparatus.

As shown in FIG. 4, a driver signal processing circuit 110 is included in the connection portion of the electronic endoscope 100. The pixel signals are input by the solid-state image sensor 108 into the driver signal processing circuit 110 in a field period. The driver signal processing circuit 110 carries out predetermined processing on the pixel signals input by the solid-state image sensor 108, and thereafter outputs the resulting image signals to the image processing circuit 220 of the processor 200.

The driver signal processing circuit 110 again accesses the memory 112 and reads out unique information of the electronic endoscope 100. The unique information of the electronic endoscope 100 recorded in the memory 112 includes, for example, the number of pixels, sensitivity, operatable field rate, model number, and the like of the solid-state image sensor 108. The driver signal processing circuit 110 outputs the unique information read out from the memory 112 to the system controller 202.

The system controller 202 performs various arithmetic operations based on the unique information of the electronic endoscope 100 and generates a control signal. The system controller 202 uses the generated control signal to control the operations and timings of the various circuits in the processor 200 such that processing that is suitable for the electronic endoscope connected to the processor 200 is performed.

The timing controller 204 supplies a clock pulse to the driver signal processing circuit 110 in accordance with the timing control performed by the system controller 202. The driver signal processing circuit 110 performs drive control on the solid-state image sensor 108 at a timing that is synchronous with the field rate of the image processed by processor 200, in accordance with the clock pulse supplied from the timing controller 204.

The image processing circuit 220, which is an example of a signal processing unit, carries out predetermined processing such as color interpolation, matrix calculation, and Y/C separation on a pixel signal input by the driver signal processing circuit 110 in a field period, and thereafter generates screen data for display on a monitor and converts the generated screen data for display on a monitor into a predetermined video format signal. The converted video format signal is output to the monitor 300. Accordingly, an image of the subject is displayed on the display screen of the monitor 300.

Also, the image processing circuit 220 includes an analysis processing circuit 230. In the special observation mode, the analysis processing circuit 230 performs spectrometric analysis processing (signal processing) based on the acquired pixel signals, calculates the value of an index correlated with biological information of the biological tissue, such as the blood amount or the oxygen saturation level, and generates image data for visually displaying the calculation result.

As described above, the electronic endoscope system 1 of the present embodiment is configured to operate in three modes, namely a normal observation mode in which the optical filter 262 is not used and the white light (normal light Ln) irradiated from the lamp 208 is used as the illuminating light, a special observation mode in which spectrometric analysis is performed using filtered light Lf as the illuminating light, the filtered light Lf being obtained due to the white light being passed through the optical filter 262, and a baseline measurement mode for obtaining correction values for special observation. The switching of the modes is performed through a user operation on the operation portion of the electronic endoscope 100 or the operation panel 214 of the processor 200.

In the normal observation mode, the system controller 202 controls the optical filter apparatus 260 to cause the optical filter 262 to retract from the optical path, irradiates the normal light Ln to the subject, and performs image capture. Also, after the image processing is carried out as needed, the image data captured using the solid-state image sensor 108 is converted into a video signal and is displayed on the monitor 300.

In the special observation mode and the baseline measurement mode, the system controller 202 controls the optical filter apparatus 260 to arrange the optical filter 262 on the optical path, irradiates the filtered light Lf to the subject, and performs image capture. Also, in the special observation mode, later-described analysis processing is performed based on the image data captured using the solid-state image sensor 108.

The baseline measurement mode is a mode in which, before the actual endoscope observation is performed, image capture is performed under illumination by the filtered light Lf, with a color reference plate such as a neutral-color diffusion plate or a reference reflection plate used as the subject, and data that is to be used in later-described standardization processing of the special observation mode is acquired.

Primary-color image data R(x,y), G(x,y), and B(x,y) obtained by performing image capture using the filtered light Lf in the baseline observation mode is stored in an internal memory of the analysis processing circuit 230 as baseline image data BL_{R}(x,y), BL_{R}(x,y), and BL_{B}(x,y) respectively. Note that R(x,y), G(x,y), B(x,y), BL_{R}(x,y), BL_{R}(x,y), and BL_{B}(x,y) are values of image data and baseline image data of the pixel (x,y). Also, the pixel (x,y) is specified by a coordinate x in the horizontal direction of the image capture surface of the solid-state image sensor 108 and a coordinate y in the vertical direction.

### Configuration and Characteristic of Optical Filter

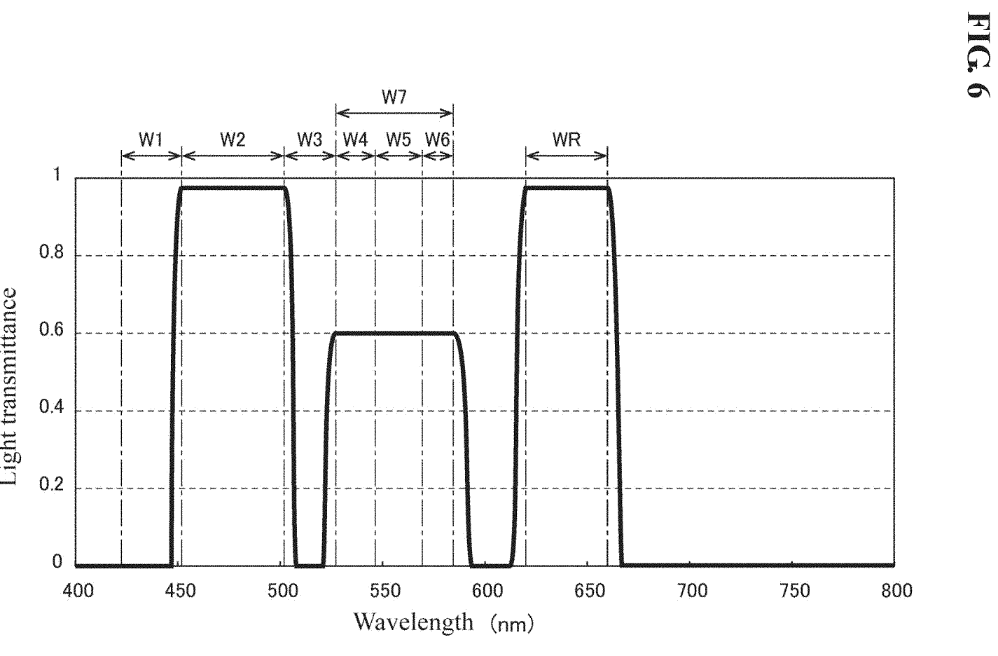

FIG. 6 is a transmission spectrum of the optical filter 262. The optical filter 262 is a single dielectric multilayer filter having an optical characteristic of selectively allowing transmission of only light in the three wavelength regions W2, W7, and WR, in at least the visible range. Although the optical filter 262 has a flat transmission characteristic in the wavelength regions W2, W7, and WR, the transmittance in the wavelength region W7 is set to be lower than in the other wavelength regions W2 and WR. This is because the light emission spectrum of the lamp 207 used in the present embodiment has a peak in the wavelength region W7, and therefore the transmittance in the wavelength region W7 is reduced so as to make the amounts of light in the wavelength regions W2, W7, and WR after transmitting through the optical filter 262 approximately uniform. The transmittance of the filter can be determined in view of the spectral characteristic of the light source that is actually used, and the sensitivity characteristic of the solid-state image sensor 108. Note that the optical filter 262 is not limited to a transmissive optical filter that transmits the illuminating light L. For example, a reflective optical filter that selectively reflects only the light of the three wavelength regions W2, W7, and WR may be used as the optical filter 262. Alternatively, an absorptive optical filter that absorbs light outside of the three wavelength regions W2, W7, and WR may be used as the optical filter 262.

### Analysis Processing in Special Observation Mode

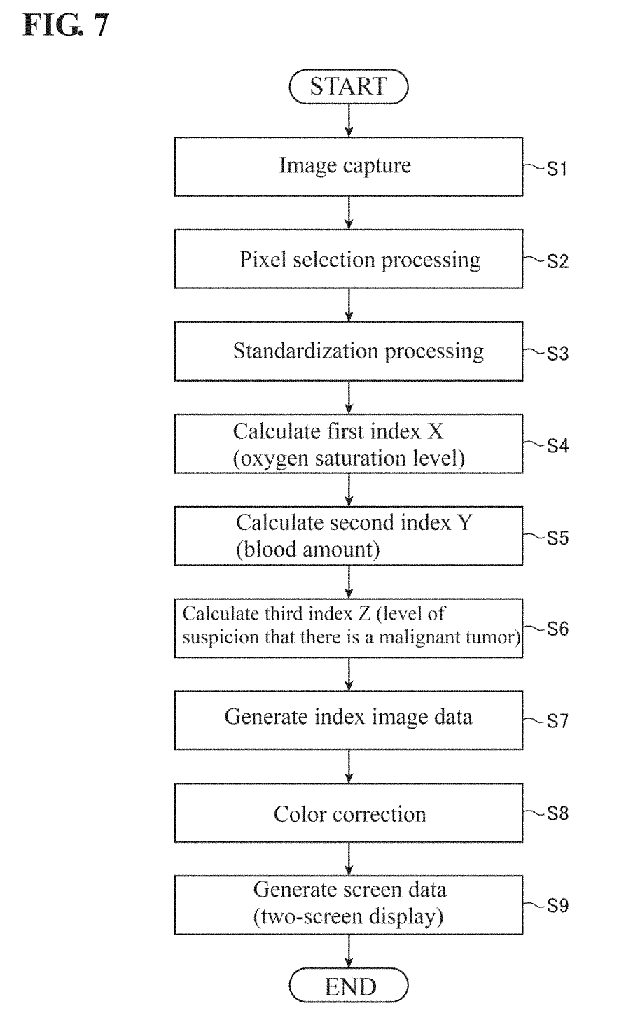

Next, analysis processing (signal processing) that is performed by the analysis processing circuit 230 in the special observation mode will be described. In the present analysis processing, analysis of biological information of biological tissue that is the subject is performed. Specifically, an index indicating the blood (hemoglobin) content of the biological tissue, and an index indicating the oxygen saturation level (percentage of the hemoglobin that is made up of oxidized hemoglobin) are calculated. FIG. 7 is a flowchart illustrating analysis processing.

In processing step S1, processing for capturing an image of the subject using the solid-state image sensor 108 is performed, and primary-color image data R(x,y), G(x,y), and B(x,y) are input to the analysis processing circuit 230.

In processing step S2, the analysis processing circuit 230 uses the input image data R(x,y), G(x,y), and B(x,y) to perform pixel selection processing for selecting the pixels (x,y) that are to be subjected to analysis processing (processing steps S3 to S6) below.

In the biological tissue that is the subject, image data at a location that does not contain blood and at a location in which the color of the biological tissue is predominantly influenced by a substance other than hemoglobin is merely noise since a significant value cannot be obtained by calculating the blood amount and the oxygen saturation level based on the color information obtained from the image data. If this kind of noise is calculated and provided to a doctor, it not only hinders suitable diagnosis, but has the adverse effect of reducing the processing speed by applying a needless load to the analysis processing circuit 230. In view of this, in the present embodiment, the pixels that are suitable for the analysis processing (i.e., pixels in which the spectral characteristic of blood is recorded) are selected and the analysis processing is performed only on the selected pixels.

In the pixel selection processing S2, only pixels for which the image data satisfies all of the following Equations 2, 3, and 4 are selected as target pixels for analysis processing.

B(x,y)/G(x,y)>a1 Equation 2

R(x,y)/G(x,y)>a2 Equation 3

R(x,y)/B(x,y)>a3 Equation 4

Here, a1, a2, and a3 are positive constants.

The above-described three conditional equations are set based on the value magnitude relationship “G component<B component<R component” in the transmission spectrum of the blood. Note that the pixel selection processing S2 may also be performed using only one or two of the above-described three conditional equations (e.g., using only equation 3 and/or equation 4 with attention given to the red color unique to blood).

In processing step S3, standardization processing is performed on the image data of the pixels selected in the pixel selection processing S2. The standardization processing S3 of the present embodiment is processing for enabling quantitative analysis by correcting the optical characteristic of the electronic endoscope system 1 itself (e.g., the transmittance of the optical filter 262 or the light sensitivity of the solid-state image sensor 108).

In the standardization processing, the analysis processing circuit 230 uses the following equation 5 to calculate the standardized image data Rs(x,y) based on the image data R(x,y) and the baseline image data BL_{R}(x,y) acquired using the filtered light Lf that passed through the optical filter 262.

Rs(x,y)=R(x,y)/BL_{R}(x,y) Equation 5

Similarly, the standardized image data Gs(x,y) and Bs(x,y) are calculated using the following Equations 6 and 7.

Gs(x,y)=G(x,y)/BL_{G}(x,y) Equation 6

Bs(x,y)=B(x,y)/BL_{B}(x,y) Equation 7

Note that in the following description, the standardized image data Rs(x,y), Gs(x,y), and Bs(x,y) are used, but the indices may be calculated using the image data R(x,y), G(x,y), and B(x,y) instead of the standardized image data Rs(x,y), Gs(x,y), and Bs(x,y) without performing the standardization processing.

In the processing step S4, the first index X that is correlated with the oxygen saturation level is calculated using the following equation 8.

X=Bs(x,y)/Gs(x,y) Equation 8

The image data Gs(x,y) indicates an optical image formed by the light in the wavelength region W7 that has transmitted through the optical filter 262. Also, the image data Bs(x,y) indicates an optical image formed by the light in the wavelength region W2 that has transmitted through the optical filter 262. As described above, the reflectance of the light in the wavelength region W2 reflected by the biological tissue (i.e., the value of the image data Bs(x,y)) depends on both the oxygen saturation level and the blood amount. On the other hand, the reflectance of the light in the wavelength region W7 reflected by the biological tissue (i.e., the value of the image data Gs(x,y)) depends not on the oxygen saturation level but on the blood amount. For this reason, by dividing the standardized reflectance Bs(x,y) by the standardized reflectance Gs(x,y), it is possible to obtain an index in which the contribution of the blood amount is canceled out. Also, due to this division, the contribution of the surface state of the biological tissue and the contribution of the incidence angle of the illuminating light (filtered light Lf) on the biological tissue can also be canceled out and an index having only the contribution of the oxygen saturation level can be obtained. Accordingly, the first index X is a good index for the oxygen saturation level.

In the processing step S5, the second index Y that is correlated with the blood amount in the biological tissue is calculated using the following equation 9.

Y=Gs(x,y)/Rs(x,y) Equation 9

As described above, the standardized reflectance Gs(x,y) is a value that depends not on the oxygen saturation level but on the blood amount. On the other hand, the standardized reflectance Rs (i.e., the value of the image data Rs) is the reflectance of the light in the wavelength region WR reflected by the biological tissue, which is hardly absorbed at all by the blood, and therefore depends on neither the oxygen saturation amount nor the blood amount. For this reason, by dividing the standardized reflectance Gs by the standardized reflectance Rs, the contribution of the surface state of the biological tissue and the contribution of the incidence angle of the illuminating light (filtered light Lf) on the biological tissue can be canceled out and an index having only the contribution of the blood amount can be obtained. Accordingly, the second index Y is a good index for the blood amount.

In processing step S6, a third index Z indicating a result of performing a logic operation on the oxygen saturation level and the blood amount is calculated based on the first index X and the second index Y.

For example, it is known that in the tissue of a malignant tumor, the blood amount is greater than in normal tissue due to angiogenesis, and the oxygen saturation level is lower than in normal tissue due to significant metabolism of oxygen. In view of this, the analysis processing circuit 230 extracts pixels in which the first index X indicating the oxygen saturation level calculated using equation 8 is smaller than a predetermined reference value (first reference value) and the second index Y indicating the blood amount calculated using equation 9 is greater than a predetermined reference value (second reference value), sets the value of the third index Z of the extracted pixels to “1”, indicating that there is suspicion of a malignant tumor, and sets the third index Z of the other pixels to “0”.

Also, the first index X, the second index Y, and the third index Z are binary indices, and the third index Z may be calculated as a logical product or a logical sum of the first index X and the second index Y. In this case, for example, Z can be calculated using Z=X·Y (logical product) or Z=X+Y (logical sum), assuming that X=1 (the oxygen saturation level is lower than a normal value) if the value on the right side of Equation 8 is less than a first reference value, X=0 (the oxygen saturation level is the normal value) if the value on the right side of Equation 8 is greater than or equal to the first reference value, Y=1 (the blood amount is greater than the normal value) if the value on the right side of Equation 9 is greater than or equal to a second reference value, and Y=0 (the blood amount is the normal value) if the value on the right side of Equation 9 is less than the second reference value.

The foregoing is an example of a case in which the third index Z is set as a binary index, but the third index Z may also be a multiple-value (or a continuous value such as a real number) index indicating the degree of suspicion that there is a malignant tumor. In this case, for example, the third index Z(x,y) indicating the degree of suspicion that there is a malignant tumor can be calculated based on the deviation from the first reference value or the average value of the first index X(x,y) and the deviation from the second reference value or the average value of the second index Y(x,y). The third index Z(x,y) can be calculated as the sum (or weighted average) or product of the deviation of the first index X(x,y) and the deviation of the second reference value.

In processing step S7, index image data is generated in which the first index X(x,y), the second index Y(x,y), or the third index Z(x,y) designated by the user is used as the pixel values (luminance value). Note that in the present processing step S7, the index image data of all of (or two of) the first index X(x,y), the second index Y(x,y), and the third index Z(x,y) may be generated.

In processing step S8, color correction processing is performed on the image data R(x,y), G(x,y), and B(x,y). The filtered light Lf that passed through the optical filter 262 includes the primary-color spectral components R (wavelength region WR), G (wavelength region W7), and B (wavelength region W2), and therefore the filtered light Lf can be used to capture a color endoscope image. However, since the spectrum of the filtered light Lf has limited bands, the image captured using the filtered light Lf sometimes has an unnatural tint compared to that of an image captured using the normal light Ln. In view of this, in the present processing step S8, color correction processing for bringing the tint closer to that of an image obtained when using the normal light Ln is performed on the image data R(x,y), G(x,y), and B(x,y), which was captured using the filtered light Lf. Accordingly, an image captured artificially using the normal light Ln (artificial normal observation image) can be obtained.

The color correction processing S8 is performed by adding or multiplying the pre-acquired correction values C_{R}, C_{G}, and C_{B}. to the image data R(x,y), G(x,y), and B(x,y). Alternatively, a color matrix Mf may be prepared and color correction may be performed using a color matrix operation. The correction values C_{R}, C_{G}, and C_{B} and the color matrix Mf are set in advance based on the image data obtained by capturing an image of a color reference plate illuminated with the filtered light Lf for example, using the electronic endoscope system 1, and are stored in the internal memory of the analysis processing circuit 230. It is also possible to perform a setting such that the color correction processing S8 is not performed.

In processing step S9, screen data to be displayed on the monitor 300 is generated based on the image data obtained by performing the color correction processing S8, the index image data generated in the processing S7, and the like. In the image data generation processing S9, for example, various types of screen data for multiple screen display, in which an endoscope image (artificial normal observation image) and one or more types of index images are displayed side by side on one screen, endoscope image display, in which only an endoscope image is displayed, index image display, in which only one or more types of index images designated by the user are displayed, and the like can be generated. The type of screen data to be generated is selected through a user operation on the operation portion of the electronic endoscope 100 or the operation panel 214 of the processor 200.

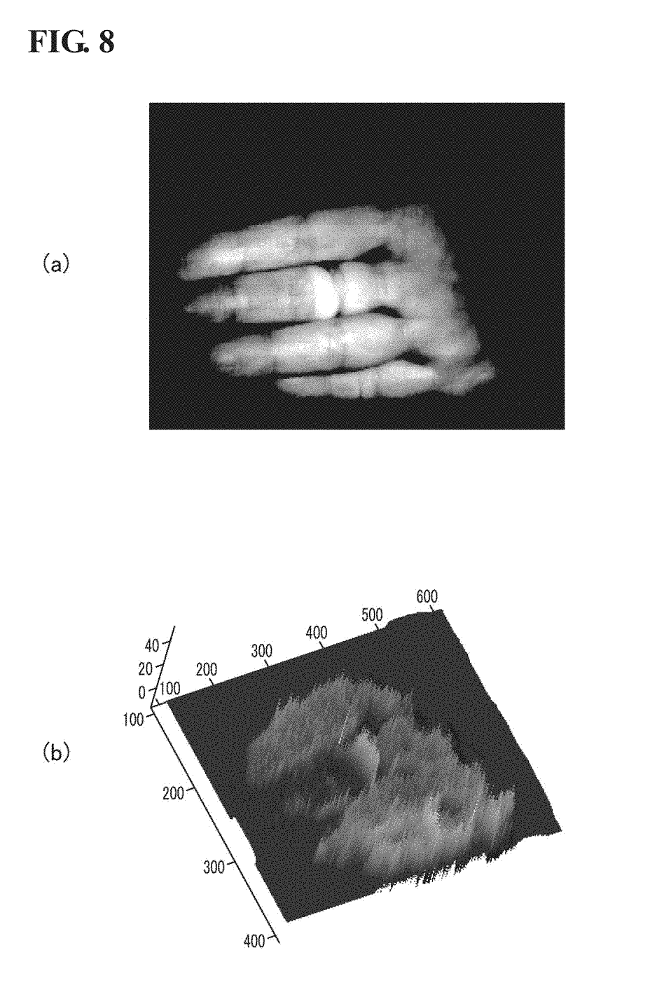

FIG. 8 is an example of a screen displayed on the monitor 300. FIG. 8(a) is an endoscope image and FIG. 6(b) is an index image of the first index X(x,y), which indicates the oxygen saturation level. Note that the image in FIG. 8 is obtained by observing a right hand in a state in which the vicinity of the proximal interphalangeal joint of the middle finger is compressed with a rubber band. FIG. 8(b) shows that the oxygen saturation level is lower on the distal side with respect to the compression portion on the right middle finger due to blood flow being hampered by the compression. Also, it can be understood that arterial blood accumulates and the oxygen saturation level is locally high near the proximal side of the compression portion.

In the special observation mode, it is possible to more reliably find a malignant tumor with characteristic changes in the blood amount and the oxygen saturation level by performing endoscope observation while performing two-screen display of the endoscope image and the index image on the monitor 300. Also, when a location that is suspected of being a malignant tumor is found, a rapid switch is made from the special observation mode to the normal observation mode through an operation of the electronic endoscope 100, all-screen display of a normal observation image with a higher image reproduction ability can be performed, and a more accurate diagnosis can be performed. The electronic endoscope system 1 of the present embodiment is configured to be able to easily and rapidly switch between the normal observation mode and the special observation mode by merely changing the method of image processing by automatically inserting and removing the optical filter 262 into and from the optical path through an operation of the electronic endoscope 100.

Also, in the electronic endoscope system 1 of the present embodiment, a configuration is employed in which the optical filter 262 that separates the three wavelength regions W2, W7, and WR is employed and the three wavelength regions W2, W7, and WR respectively transmit through the B filter, G filter, and R filter of the solid-state image sensor 108. According to these configurations, it is possible to generate an endoscope image and an index image of one frame through image capture of one frame (two fields). For this reason, image data of multiple frames is not used in the calculation of the oxygen saturation level as in the endoscope system disclosed in Patent Document 2, and therefore it is possible to display the endoscope image and the index image simultaneously without causing a problem such as a reduction of the frame rate of the captured image in the special observation mode.

Also, according to the present embodiment, the optical filter 262 has the characteristic of allowing light of the three wavelength regions W2, W7, and WR to pass. For this reason, in the special observation mode, it is not necessary to sequentially insert multiple optical filters having different transmission characteristics into the optical path of the illuminating light. For example, if one of three optical filters, namely an optical filter that transmits only the light of the wavelength region W2, an optical filter that transmits only the light of the wavelength region W7, and an optical filter that transmits only the light of the wavelength region WR is selectively inserted into the optical path of the illuminating light to acquire the image data, a movement mechanism for moving the optical filters synchronously with the frame rate is needed. Also, providing this kind of movement mechanism can bring about a disadvantage in that the processor 200 has a comparatively larger size, greater complexity, and lower durability due to the movement mechanism including a movable portion. Furthermore, if multiple optical filters are sequentially inserted into the optical path of the illuminating light, the light amount of the illuminating light emitted to the subject varies each time the optical filter traverses the optical path and image data with a stable brightness is not obtained in some cases. However, according to the present embodiment, it is not necessary to drive the optical filter 262 during the image capture processing in the special observation mode, and therefore it is possible to suppress an increase in the size and a reduction of the durability of the processor 200 (light source apparatus) and variation in the light amount of the illuminating light.

Note that the transmission spectrum of the optical filter 262 in the present embodiment is not limited to that shown in FIG. 6. The light amount (specifically, the maximum light transmittance) of the light in the wavelength regions W2, W7, and WR that passes through the optical filter 262 can change according to the transmission spectrum of the on-chip color filters (R filter, G filter, and B filter) of the solid-state image sensor 108. For example, the light transmittance of the G filter of the solid-state image sensor 108 is lowered, whereas the light transmittance of the wavelength region W7 of the optical filter 262 can be increased.

Also, in the case where the oxygen saturation level is calculated using the image data of two frames as with the endoscope system disclosed in Patent Document 2, if the subject moves with respect to the solid-state image sensor, the position of the subject in the captured image changes between the two frames in some cases. In this case, a case may occur in which the oxygen saturation level cannot be correctly calculated using the image of two frames or the edges of the subject image are emphasized. In contrast to this, according to the present embodiment, biological information such as the oxygen saturation level is calculated using a captured image of one frame. For this reason, even if the subject moves during image capture processing, the indices indicating the biological information can be calculated correctly without the edges being emphasized.

The foregoing is a description of an illustrative embodiment of the present invention. The embodiment of the present invention is not limited to the above description, and various modifications are possible in the technical idea of the present invention. For example, content obtained by combining the embodiment and the like specified illustratively in the present specification, or an obvious embodiment or the like is also included in the embodiment of the present application.

For example, the first embodiment is an example in which the wavelength region W2 is used as the wavelength region of the blue color used in the special observation mode, but it is also possible to use the wavelength region W1 instead of the wavelength region W2. As shown in FIG. 1, in the wavelength region W1, the difference in light transmittance T (i.e., absorption) between the oxidized hemoglobin and the reduced hemoglobin is greater than in the wavelength region W2. For this reason, by using the wavelength region W1, it is possible to detect a change in the oxygen saturation level with greater sensitivity.

Also, the above-described embodiment is an example in which spectral analysis results are displayed using a grayscale or monochrome index image, but the method for displaying the analysis results is not limited to this. For example, it is also possible to use a configuration in which a modification is added to the image data R(x,y), G(x,y), and B(x,y) according to the index value. For example, processing for increasing the brightness, processing for changing the hue (e.g., processing for increasing the R component to strengthen the redness or processing for rotating the hue by a predetermined angle), and processing for causing pixels to blink (or for periodically changing the hue) can be performed on the pixels whose index values have exceeded a reference value.

### Second Embodiment

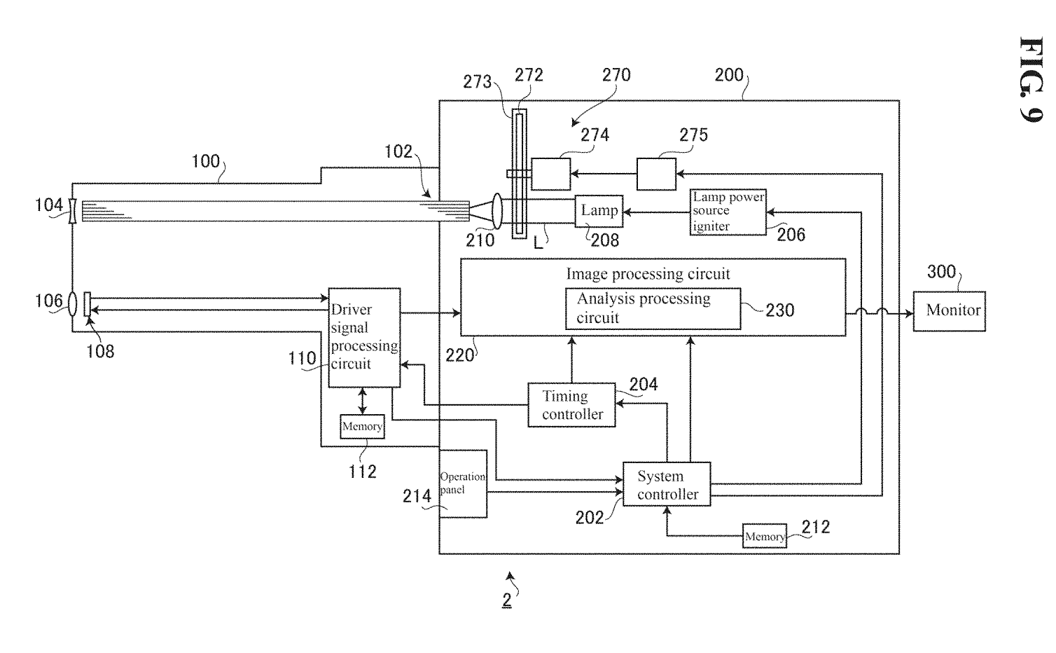

Next, a second embodiment of the present invention will be described. In the second embodiment, the present invention is applied to an electronic endoscope system, similarly to the first embodiment. The electronic endoscope system of the second embodiment employs a so-called surface sequential method, in which a solid-state image sensor for monochrome (grayscale) image capture, which does not have an on-chip color filter, is used. FIG. 9 is a block diagram showing a configuration of an electronic endoscope system 2 of the second embodiment. As shown in FIG. 9, the electronic endoscope system 2 of the second embodiment includes an optical filter apparatus 270. The optical filter apparatus 270 includes a rotary turret 273, a motor 274 connected to the rotary turret 273, and a motor drive circuit 275 that performs drive control on the motor 274. Here, the motor 274 and the motor drive circuit 275 constitute an example of a filter drive unit. Also, an optical filter 272 is attached to the rotary turret 270. Note that in the description below, it is assumed that identical reference numerals are used for constituent elements that are identical to those in the first embodiment, for the sake of convenience in the description.

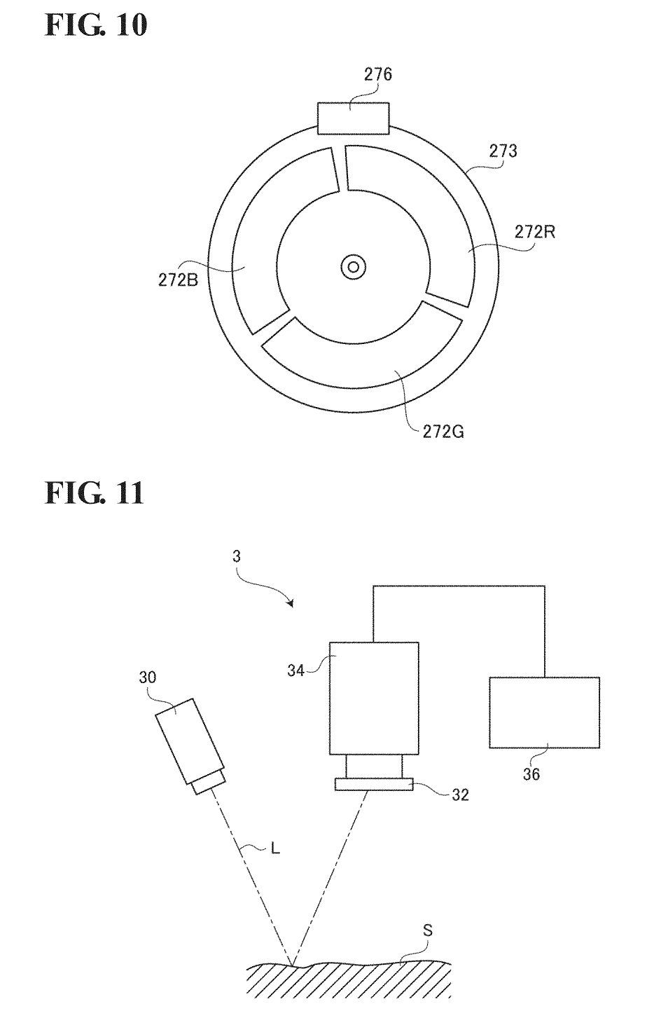

FIG. 10 is a front view of the rotary turret 273. The optical filter 272 includes a band-pass filter 272B that transmits only light of the wavelength region W2, a band-pass filter 272G that transmits only light of the wavelength region W7, and a band-pass filter 272R that transmits only light of the wavelength region WR. The band-pass filters 272B, 272G, and 272R are examples of first, second, and third band-pass filters, respectively. As shown in FIG. 10, the band-pass filters 272B, 272G, and 272R are arranged side by side in the circumferential direction of the rotary turret 273. The band-pass filters 272B, 272G, and 272R are fan-shaped and are arranged at an angular pitch (here, an angular pitch of about 120 degrees) corresponding to the frame cycle. In the present embodiment, one rotation of the optical filter 272 corresponds to one frame.

The motor drive circuit 275 drives the motor 274 according to control performed by the system controller 202. Due to the rotary turret 273 being rotated by the motor 274, the three band-pass filters 272B, 272G, and 272R are sequentially inserted into the optical path of the illuminating light. Accordingly, three types of illuminating light with different spectra are taken out from the illuminating light L emitted from the lamp 208, at timings synchronous with image capture. Specifically, during the rotation operation, the rotary turret 273 selectively takes out the illuminating light of the wavelength region W2 using the band-pass filter 272B, the illuminating light of the wavelength region W7 using the optical filter 272G, and the illuminating light of the wavelength region WR using the band-pass filter 272R. The taken-out illuminating light is emitted sequentially to the subject. The rotation position and the phase of rotation of the rotary turret 273 are controlled by detecting an opening (not shown) formed near the outer circumference of the rotary turret 273 using a photointerrupter 276.

A solid-state image sensor 108′ outputs electrical charges corresponding to the light amounts of the light received while the illuminating light of the wavelength W2 is emitted to the subject as a pixel signal B. The solid-state image sensor 108′ outputs electrical charges corresponding to the light amounts of the light received while the illuminating light of the wavelength W7 is emitted to the subject as a pixel signal G. The solid-state image sensor 108′ outputs electrical charges corresponding to the light amounts of the light received while the illuminating light of the wavelength WR is emitted to the subject as a pixel signal R. Accordingly, pixel signals R, G, and B that are used in the analysis processing described in the first embodiment can be obtained.

As described in the first embodiment, the pixel signals R, G, and B are used in both the display of the normal endoscope image and the display of the index image indicating an index of the biological information. For this reason, with the electronic endoscope system 2 of the second embodiment, it is not necessary to use a new and separate optical filter or light source apparatus in order to display the index image. For this reason, the endoscope image and the index image can be displayed simultaneously without reducing the frame rate of the captured image. Also, with the electronic endoscope system 2 of the second embodiment, the solid-state image sensor 108′ for monochrome image capture, which does not include an on-chip color filter, is used in image capture processing of a subject, and therefore a high-definition captured image can be obtained compared to the case of using a solid-state image sensor having an on-chip color filter.

### Third Embodiment

Next, a third embodiment of the present invention will be described. The first and second embodiments are examples in which the present invention is applied to an electronic endoscope system, but the present invention can also be applied to a system in which another type of digital camera (e.g., a digital single-lens reflex camera or a digital video camera) is used. In the third embodiment, the present invention is applied to an image capture system including a digital video camera. FIG. 11 is a block diagram showing a configuration of an image capture system 3 of the third embodiment. The image capture system 3 includes a light source apparatus 30, an optical filter 32, a digital video camera (image capture apparatus) 34, and a monitor 36.

The light source apparatus 30 emits illuminating light L that illuminates a subject S. The illuminating light L is mainly light (white light including at least the visible range) having a spectrum that extends from the visible range to the infrared range, which is invisible. The illuminating light L (reflected light) reflected by the subject S is incident on the optical filter 32.

The optical characteristic of the optical filter 32 is the same as the optical characteristic of the optical filter 262 of the first embodiment. That is, the optical filter 32 selectively allows transmission of only light in the three wavelength regions W2, W7, and WR. The reflected light that has passed through the optical filter 32 is incident on the image capture apparatus 34.

The image capture apparatus 34 includes a solid-state image sensor and a signal processing circuit or the like for carrying out signal processing on the pixel signals output from the solid-state image sensor. The solid-state image sensor includes so-called on-chip color filters, namely an R filter, a G filter, and a B filter, on pixels. The solid-state image sensor outputs the pixel signals R, G, and B according to the light amounts of the received reflected light.

The image capture apparatus 34 generates a normal endoscope image and an index image indicating indices of biological information, similarly to the image processing circuit 220 of the first embodiment based on the image signals R, G, and B. The endoscope image and the index image generated by the image capture apparatus 34 are displayed on the monitor 36.

In this manner, in the third embodiment, the optical filter 32 is arranged not in front in the emission direction of the illuminating light L of the light source apparatus 30 (between the light source apparatus 30 and the subject S), but in front of the solid-state image sensor (between the solid-state image sensor and the subject S). Accordingly, both the endoscope image and the index image can be displayed without changing the spectral characteristic of the illuminating light L that illuminates the subject. Also, in the third embodiment, the image capture apparatus 34 can be arranged separate from the subject S, and therefore the analysis processing performed by the image capture system 3 and the direct observation of the subject S by an operator can be performed simultaneously. In the third embodiment, the spectral characteristic of the illuminating light L is not changed during the analysis processing, and therefore the tone of the subject S can be prevented from changing due to the illuminating light L, from the perspective of an operator directly observing the subject S.

Also, in the third embodiment, the endoscope image and the index image can be generated through image capture of one frame (two fields), similarly to the first embodiment. Accordingly, since it is not necessary to switch the light source apparatus for each frame as in Patent Document 2, the endoscope image and the index image can be displayed simultaneously without causing a reduction of the frame rate.

In the first and second embodiments, the optical filters 262 and 272 are arranged in front of the light source apparatus (lamp 208), and in the third embodiment, the optical filter 32 is arranged in front of the solid-state image sensor, but there is no limitation to this. The optical filter can be arranged at any position on the optical path of the illuminating light from the light source apparatus to the solid-state image sensor.

For example, in the electronic endoscope system 1 of the first embodiment, the optical filter 262 may be arranged in front of the light dispersion lens 104, in front of the object lens 106, or between the object lens 106 and the solid-state image sensor 108. Also, in the image capture system 3 of the third embodiment, the optical filter 32 may be arranged in front of the light source apparatus 30.

Also, the optical filter may be a reflection member that can select a wavelength, and for example, may be a dichroic mirror. In this case, the dichroic mirror has a property of reflecting illuminating light used as the filtered light Lf and transmitting the light other than the filtered light Lf.

1. A calculation system, comprising:
one light source apparatus configured to emit illuminating light;
a wavelength selection unit configured to select light of at least two specific wavelength regions included in the illuminating light;
an image sensor configured to receive light from biological tissue, which is a subject, and output pixel signals corresponding to the received light; and
a signal processing unit configured to perform predetermined signal processing on the pixel signals output from the image sensor,
wherein the signal processing unit calculates an index indicating a concentration of a predetermined biological substance included in the biological tissue based on the pixel signals output from the image sensor according to the light of the at least two specific wavelength regions.

2. The calculation system according to claim 1, wherein
the image sensor has three color filters for color image capture on light receiving surfaces of pixels, and wavelength regions of two of the three colors include the two specific wavelength regions respectively.

3. The calculation system according to claim 2, wherein
the color filters include an R filter, a G filter, and a B filter, for which the wavelength regions of light that is transmitted therethrough are mutually different,
one of the specific wavelength regions, which is transmitted by the G filter, includes a wavelength region defined by two predetermined isosbestic points of the hemoglobin, and
the other of the specific wavelength regions, which is transmitted by the B filter, includes a wavelength region defined by two isosbestic points that are different from the two predetermined isosbestic points of the hemoglobin.

4. The calculation system according to claim 3, wherein
the wavelength region of light that is transmitted by the R filter includes a first wavelength region of 600 nm or more,
the wavelength region of light that is transmitted by the G filter includes a second wavelength region of 528 nm or more and 584 nm or less, and
the wavelength region of light that is transmitted by the B filter includes a third wavelength region of 452 nm or more and 502 nm or less.

5. The calculation system according to claim 3, wherein
the wavelength selection unit selects light of the first wavelength region, the second wavelength region, and the third wavelength region, which is included in the illuminating light.

6. The calculation system according to claim 2, wherein
the wavelength selection unit is a single optical filter that selectively transmits or reflects light of the at least two specific wavelength regions.

7. The calculation system according to claim 1, wherein
the wavelength selection unit includes at least two band-pass filters that respectively correspond to the at least two specific wavelength regions, and
the calculation system further comprises a filter drive unit configured to selectively insert one of the at least two band-pass filters into an optical path of the illuminating light.

8. The calculation system according to claim 7, wherein
the wavelength selection unit includes a first band-pass filter, a second band-pass filter, and a third band-pass filter,
the first band-pass filter selectively transmits light of a wavelength region of 600 nm or more,
the second band-pass filter selectively transmits light of a second wavelength region of 528 nm or more and 584 nm or less, and
the third band-pass filter selectively transmits light of a third wavelength region of 452 nm or more and 502 nm or less.

9. The calculation system according to claim 1, wherein
the wavelength selection unit is arranged between the light source apparatus and the biological tissue, and
the biological tissue is illuminated with the illuminating light selected by the wavelength selection unit as the light of the specific wavelength region.

10. The calculation system according to claim 1, wherein
the wavelength selection unit is arranged between the biological tissue and the image sensor,
the wavelength selection unit selects light of the specific wavelength from reflected light reflected by the biological tissue, and
the image sensor receives the reflected light selected by the wavelength selection unit as the light of the specific wavelength region.
